(19) United States
(12) Patent Application Publication
(10) Pub. No.: US 2005/0176123 A1
(43) Pub. Date: Aug. 11, 2005

(54) Protease inhibitor

(75) Inventor: Nobuhiko Katunuma, Tokushima (JP)

Correspondence Address: KNOBBE MARTENS OLSON & BEAR LLP, 2040 MAIN STREET, FOURTEENTH FLOOR, IRVINE, CA, 92614, US

(73) Assignee: Morinaga Milk Industry Co., Ltd., Minato-ku, Tokyo (JP)

(21) Appl. No.: 10/513,523

(22) PCT Filed: Nov. 25, 2003
(86) PCT No.: PCT/JP03/15007

(30) Foreign Application Priority Data
Nov. 29, 2002 (JP) 2002-347802

(51) Int. Cl. A61K 38/43; C12N 9/99

(52) U.S. Cl. 435/184; 424/94.1

(57) ABSTRACT

This invention relates to a cysteine protease inhibitor comprising one or more of lactoferrin, a partial peptide of lactoferrin, and transferrin, as an active ingredient. The cysteine protease inhibitor of the present invention can be used for preventive or therapeutic drug for osteoporosis, malignant hypercalcemia etc. and used for food, drink, feed and the like.

### TECHNICAL FIELD

The present invention relates to a cysteine protease inhibitor comprising one or more of components selected from the group consisting of lactoferrin, a partial peptide of lactoferrin or transferrin as an active ingredient, which can be used for preventive or therapeutic agents for osteoporosis, malignant hypercalcemia etc., and used for food, drink, feed and the like.

### BACKGROUND ART

A proteolytic enzyme having a thiol group at its active center is generically called as cysteine protease (thiol protease). Cathepsin L, cathepsin B or cathepsin K is one of typical cysteine proteases along with calcium-dependent neutral protease (CAMP), papain, ficin, promelain and the like. Substance shaving inhibitory action against those cysteine proteases are expected to be used as therapeutic agents for diseases associated with cysteine proteases including muscular dystrophy, dystrophia, myocardial infarct, apoplexy, Alzheimer's disease, disturbance of consciousness or motility caused by head trauma, multiple sclerosis, peripheral neuropathy, cataract, inflammation, allergy, fulminant hepatitis, osteoporosis, hypercalcemia, breast cancer, prostate cancer or enlarged prostate, or as a growth inhibitor or metastasis preventive agent for cancer, an antithrombotic drug and the like. Moreover, in recent years, the relationship between cathepsin L, cathepsin B, and osteoporosis or malignant hypercalcemia has been elucidated based on the studies made by Katsunuma et al. Accordingly, in particular, application of a cathepsin L inhibitor as a therapeutic agent for osteoporosis or malignant hypercalcemia is attracting attention (Nobuhiko Katsunuma, “BIO media” Vol. 7, No. 6, pp. 73 to 77, 1992). In bone tissue, osteogenesis by osteoblast and bone resorption by osteoclast are occurring throughout life. Ingrowing ages, bone mass is increased due to excessive osteogenesis over bone resorption, whereas in older ages, bone mass is decreased due to excessive bone resorption over osteogenesis, which leads to development of osteoporosis. There are various causes of osteoporosis, and especially, one of main causes thereof is bone collapse (bone resorption), which is further divided into the following two causes. That is, one is attributed to failure of absorption and deposition of calcium, more specifically related to supply, transfer, absorption and deposition of calcium, in which vitamin D derivatives, female hormone (estrogen) and the like are considered to be involved. The other one is associated with promoted degradation of collagen, a bone-supporting tissue, and a principal cause thereof is degradation of bone collagen by cysteine proteases which are secreted from the lysosome located in the osteoclast, in particular, cathepsin L, cathepsin B and cathepsin K. The cathepsin L and capthepsin B secreted from the lysosome in the osteoclast promote degradation of collagen in bone tissue, whereby old bones are caused to lyse, and calcium is freed and released into the blood together with hydroxyproline. Therefore, inhibition of cathepsin L, cathepsin B and cathepsin K-mediated collagen degradation enables prevention of excessive bone collapse, thus making it possible to treat osteoporosis. Estrogen, anabolic hormone, calcium preparation, vitamin D, calcitonin, bisphosphonate or the like is known as a therapeutic agent for such osteoporosis. In addition, development of a therapeutic agent for osteoporosis using some cysteine protease inhibitors is under progress as regards a therapeutic agent for osteoporosis having a mechanism of action of so-called cysteine protease inhibition including cathepsin L inhibition, cathepsin B inhibition and cathepsin K inhibition (JP 07-179496 A, JP 2002-501502A). However, further development of therapeutic agents for osteoporosis has been desired.

Meanwhile, hypercalcemia is a metabolic disorder where calcium concentration in serum is elevated beyond the normal value, and is often seen in patients with tumor. It is said that, if hypercalcemia is neglected, the life of the patients would be 10 days long at most. In many cases, it is caused due to bone metastasis of tumor. When tumor transfers to bone, bone collapse occurs and calcium is released into the blood. The released calcium is disposed in the kidney, and hypercalcemia develops when speed of bone collapse exceeds the disposing capacity of the kidney. As a method of treatment, a method of promoting calcium excretion from the kidney by use of infusion of physiological saline with furosemide and a method of using calcitonin as a therapeutic agent for osteoporosis are known. Namely, it is said that a therapeutic agent for osteoporosis which suppresses bone resorption can be also effective as a therapeutic agent for malignant hypercalcemia.

The followings have already been disclosed as cysteine protease inhibitors which may be used for such purposes, by the inventors of the present invention.
(1) Cathepsin L-specific inhibitory polypeptide (JP 07-179496 A)
(2) Thiol protease inhibitor (JP 09-221425 A)
(3) Valine derivative and its use (JP 2001-139534 A)
(4) Thiol protease inhibitor (JP 07-242600 A)
(5) FA-70C1 substance (JP 2000-72797 A)
(6) FA-70D substance, its production method and its use (WO 97/31122)

However, further development of cysteine protease inhibitors which are antigen-free and can be used as safe material has been desired.

On the other hand, it has been known so far that protease inhibitory substances are present in breast milk. The known protease inhibitory substances contained in breast milk include α1-antichymotrypsin and α1-antitrypsin, and inhibitors including inter α2-trypsin inhibitory substance, α2-antiplasmin, α2-macroglobulin, antithrombin III and antileukoprotease are contained in a minute amount in breast milk (Isao Kiyosawa, “Human Milk in Infant Nutrition” Kanehara & Co., Ltd., pp. 80 to 81).

The following proteins having cysteine protease inhibitory activity in milk have already been disclosed.
(1) Novel cysteine protease inhibitor with a molecular weight of approximately 57 kDa which has a sugar chain and is derived from bovine colostrum (JP 07-2896 A).
(2) Novel cysteine protease inhibitor with a molecular weight of 16±2 kDa or 13±2 kDa which is derived from colostrum (JP 07-126294 A).
(3) Novel protein with a molecular weight of 16±2 kDa or 13±2 kDa which is derived from human milk, and a production method for the same (JP 10-80281 A).
(4) Bone resorption inhibitor which includes basic cystatin of milk origin which is prepared from milk and/or degradation product of the basic cystatin of milk origin, as an active ingredient (JP 2000-281587 A).
(5) Cystatin C contained in milk basic protein (MBP), and a bone resorption inhibitory effect by the cystatin C in vitro (“Bioscience, Biotechnology, and Biochemistry, Japan” Vol. 66, No. 12, 2002, pp. 2531 to 2536).

Proteins contained in mammalian milk in a large amount include lactoferrin and β-casein. Lactoferrin (hereinafter, sometimes abbreviated to Lf) is an iron-bound glycoprotein which is mainly contained in breast milk and has molecular weight of approximately 80 kDa, and it is known to exhibit antibacterial effects on pathogenic microorganisms including Escherichia coli, Candida, Clostridium and Staphylococcus (Journal of Pediatrics, vol. 94, page 1, 1979 and Journal of Dairy Science, vol. 67, page 60, 1984). Further, it is widely used for treatment of diseases, as a milk protein having various activities. Lactoferrin, a protein derived from milk, has high safety level and can be continuously used for a long term. Further, lactoferrin itself has no flavor and no smell and therefore, it is versatile as an additive into various foods, pharmaceuticals and feed.

As examples of therapeutic agents to which lactoferrin is applied, the followings are disclosed.
(1) Anti-tumor agent (JP 05-86932 B)
(2) Anti-rheumatoid agent (JP 05-186368 A)
(3) IgA production-accelerating agent (JP 06-32743 A)
(4) Processed milk powder for baby nursing effective for improving state of feces. (JP 06-205640 A)
(5) Therapeutic agent for angiogenesis-related disease (JP09-194388 A)
(6) Oral agent for inhibiting cancer metastasis (JP 10-59864)
(7) IgE production-inhibitor (JP 2001-158748 A)

As effects of hydrolysate of Lf, the followings are disclosed.
(8) Antibacterial effect and tyrosinase-inhibitory effect (European Patent 438750)
(9) Inhibitory effect on attachment of pathogenic bacteria to cells (JP 03-220130 A)
(10) Antivirus effect (JP 01-233226 A)

Further, it is disclosed that bone-enriching agent comprising iron-bound lactoferrin as an active ingredient is effective for preventing or treating bone-related diseases (JP 2000-281586 A). This is a technology where iron, a coenzyme of an enzyme involved in hydration of proline and lysine which are precursors of bone substrate collagen, is provided by administering iron-lactoferrin, namely, iron-bound lactoferrin, as an active ingredient, and thereby, collagen-synthesis is enhanced.

As describe above, cysteine protease-inhibitory substances have been thus far found and they have also been found in mammalian cells, blood, urine and milk. Among cysteine protease-inhibitory substances, substances derived from proteins are generally called cystatin. It is known that the cystatin family has common active site, and the sequence is disclosed (Hayaishi Osamu wrote, “proteases and inhibitors thereof,” Medical View Co. Ltd. Eds., 1^{st} edition, 1^{st} printing, pages 104-115, 1993).

However, it has not been known that lactoferrin, partial peptide thereof and transferrin have cysteine protease inhibitory activity. Further, neither had it been known that lactoferrin and transferrin have a region homologous to the sequence of the active site preserved in cystatin family. Moreover, agents for preventing or treating bone-related diseases, which have a mechanism of action of inhibiting cystein protease activity by lactoferrin, partial peptide thereof or transferrin, has not been known.

### DISCLOSURE OF THE INVENTION

An object of the present invention is to provide a versatile cysteine protease inhibitor which can be widely used as food materials, and which can be used for preventive or therapeutic agents for osteoporosis, malignant hypercalcemia and the like, and used for various types of food, drink and feed.

As a result of extensive studies for searching a cysteine protease inhibitor which may be used as antigenic-free and safe material, the inventors of the present invention found that lactoferrin which is a protein derived from milk, a peptide fragment derived from lactoferrin, and transferrin have cysteine protease inhibitory activity, and thereby completed the present invention.

The gist of the present invention is as in the following (1) to (12).
(1) A cysteine protease inhibitor which comprises one or more of lactoferrin, a partial peptide of lactoferrin, and transferrin, as an active ingredient.
(2) The cysteine protease inhibitor according to (1), wherein lactoferrin and/or transferrin are one or more of metal-saturated form, partially metal-saturated form and apo form.
(3) The cysteine protease inhibitor according to (1) or (2), wherein lactoferrin and/or transferrin are one or more of the proteins or peptides as shown in the following (a) to (d);
(a) a protein or peptide having amino acid sequence of at least amino acid numbers 679 to 695 of the amino acid sequence shown in SEQ ID No. 1 of the Sequence Listing,
(b) a protein or peptide having amino acid sequence of at least amino acid numbers 679 to 695 of the amino acid sequence shown in the SEQ ID No. 1 of the Sequence Listing, including substitution, deletion, addition or inversion of one or plural amino acids, and having cysteine protease inhibitory activity,
(c) a protein or peptide having amino acid sequence of at least amino acid numbers 676 to 692 of the amino acid sequence shown in SEQ ID No. 2 of the Sequence Listing, and
(d) a protein or peptide having amino acid sequence of at least amino acid numbers 676 to 692 of the amino acid sequence shown in the SEQ ID No. 2 of the Sequence Listing, including substitution, deletion, addition or inversion of one or plural amino acids, and having cysteine protease inhibitory activity.
   (4) The cysteine protease inhibitor according to any one of (1) to (3), which is a preventive or therapeutic agent for a disease associated with cysteine protease.
   (5) The cysteine protease inhibitor according to (4), wherein the disease associated with cysteine protease is osteoporosis or malignant hypercalcemia.
   (6) A food and drink composition or feed composition, which is produced by adding the cysteine protease inhibitor according to any one of (1) to (5).
   (7) A method for treating a disease associated with cysteine protease, wherein the cysteine protease inhibitor according to any one of (1) to (6) is administered to a subject.
   (8) A use of one or more of lactoferrin, a peptide fragment of lactoferrin, and transferrin in manufacture of a cysteine protease inhibitor.
   (9) The use according to (8), wherein lactoferrin and/or transferrin are one or more of metal-saturated form, partially metal-saturated form and apo form.
   (10) The use according to (8) or (9), wherein lactoferrin and/or transferrin are one or more of the proteins or peptides as shown in the following (a) to (d);
(a) a protein or peptide having amino acid sequence of at least amino acid numbers 679 to 695 of the amino acid sequence shown in SEQ ID No. 1 of the Sequence Listing,
(b) a protein or peptide having amino acid sequence of at least amino acid numbers 679 to 695 of the amino acid sequence shown in the SEQ ID No. 1 of the Sequence Listing, including substitution, deletion, addition or inversion of one or plural amino acids, and having cysteine protease inhibitory activity,
(c) a protein or peptide having amino acid sequence of at least amino acid numbers 676 to 692 of the amino acid sequence shown in SEQ ID No. 2 of the Sequence Listing, and
(d) a protein or peptide having amino acid sequence of at least amino acid numbers 676 to 692 of the amino acid sequence shown in the SEQ ID No. 2 of the Sequence Listing, including substitution, deletion, addition or inversion of one or plural amino acids, and having cysteine protease inhibitory activity.
   (11) The use according to any one of (8) to (10), wherein the cysteine protease inhibitor is a preventive and therapeutic agent for a disease associated with cysteine protease.
   (12) The use according to (11), wherein the disease associated with cysteine protease is osteoporosis or malignant hypercalcemia.

### BRIEF DESCRIPTION OF THE DRAWINGS

FIG. 1 is a diagram (photograph) which shows detection of bovine milk protein by reverse zymography.

FIG. 2 is a diagram which shows inhibitory activities of human lactoferrin to cysteine proteases.

FIG. 3 is a diagram which shows amino acid sequences of a partial peptide of human lactoferrin (human lactoferrin peptide Y679-K695) which was produced in Production Example 1, a partial peptide of bovine lactoferrin (bovine lactoferrin peptide Y676-K692), a peptide having amino acid sequence of amino acid numbers 666-682 in the amino acid sequence shown in SEQ ID No. 3 of the Sequence Listing (human transferrin peptide Y666-R682) and a peptide having amino acid sequence of amino acid numbers 672-688 in the amino acid sequence shown in SEQ ID No. 4 of the Sequence Listing (bovine transferrin peptide Y672-R688).

FIG. 4 is a diagram which shows inhibitory activities of human lactoferrin peptide Y679-K695 to cysteine proteases.

FIG. 5 is a diagram which shows inhibitory activities of transferrin to cysteine proteases.

### BEST MODE FOR CARRYING OUT THE INVENTION

Preferred embodiments of the present invention will be described in detail below. However, the present invention is not limited to the following preferred embodiments, and any changes may be made within the scope of the present invention. All percentages used in the present specification are represented by mass unless otherwise noted.

The present invention relates to a cysteine protease inhibitor comprising one or more of lactoferrin (e.g., human lactoferrin having amino acid sequence of Swiss-Prot Accession No.: P02788, bovine lactoferrin having amino acid sequence of Swiss-Prot Accession No.: P24627, goat lactoferrin having amino acid sequence of Swiss-Prot Accession No.: Q29477, horse lactoferrin having amino acid sequence of Swiss-Prot Accession No.: 077811), a partial peptide of lactoferrin, transferrin (e.g., human transferrin having amino acid sequence of Swiss-Prot Accession No.: P02787, bovine transferrin having amino acid sequence of Swiss-Prot Accession No.: Q29443, horse transferrin having amino acid sequence of Swiss-Prot Accession No.: P27425), as an active ingredient.

Lactoferrin used in the present invention may be commercially available lactoferrin, or lactoferrin which can be isolated according to conventional procedures including ion-exchange chromatography from raw materials such as mammalian colostrum, transitional milk, matured milk, last-phase milk, or skim milk or whey, which are processed milk. In particular, it is preferable to use commercially available lactoferrin (e.g. manufactured by Morinaga Milk Industry Co., Ltd.) which is produced at an industrial scale. In addition, lactoferrin produced by genetic engineering using microorganisms, mammalian cells or transgenic animals etc. can be used.

Metal content in lactoferrin is not particularly limited for the effect of the cystein protease inhibitor of the present invention, and one or more of the groups consisting of apo-form lactoferrin which is deironized with hydrochloric acid or citric acid etc., metal-saturated lactoferrin having saturation degree of 100% which is obtainable by chelating the apo-form lactoferrin with metals including iron, copper, zinc, manganese etc., and lactoferrin partially saturated with metal in which metal is bound at saturation degree of less than 100% can be used.

As production methods of partial peptide of lactoferrin used in the present invention, there can be exemplified a production method by hydrolyzing the above-mentioned lactoferrin with acid or protease according to conventional procedures, a production method by producing recombinant peptides according to genetic engineering or a method of producing synthetic peptides according to chemical synthesis. When the production method by hydrolysis is applied, the partial peptide of lactoferrin of the present invention can be used as a mixture in which partial peptide is contained in hydrolysate, or can be used as a peptide purified according to conventional procedures including HPLC etc.

Preferable forms of lactoferrin or partial peptide of lactoferrin used in the present invention can be exemplified as a human lactoferrin having amino acid sequence shown in SEQ ID No. 1 of the Sequence Listing or protein or peptide having amino acid sequence of at least amino acid numbers 679-695 of the amino acid sequence shown in SEQ ID No. 1 of the Sequence Listing. There can also be exemplified a bovine lactoferrin having amino acid sequence shown in SEQ ID No. 2 of the Sequence Listing or protein or peptide having amino acid sequence of at least amino acid numbers 676-692 of the amino acid sequence shown in SEQ ID No. 2 of the Sequence Listing. Note that sequences of amino acid numbers 1 to 19 of the amino acid sequence shown in SEQ ID No. 1 of the Sequence Listing and the amino acid sequence shown in SEQ ID No. 2 of the Sequence Listing are signal sequences.

These lactoferrin and partial peptide of lactoferrin have cysteine protease inhibitory activity, and therefore they can be used for the cysteine protease inhibitor of the present invention.

Moreover, it is considered that peptides having amino acid sequence which includes amino acid numbers 679 to 695 of SEQ ID No. 1 in the Sequence Listing and further extends to one or both of the N-termius side and the C-termius side, and peptides having amino acid sequences which includes amino acid numbers 676 to 692 of SEQ ID No. 2 in the Sequence Listing and further extends to one or both of the N termius side and the C termius side, have cysteine protease inhibitory activity, because full-length lactoferrin has cysteine protease inhibitory activity as described later.

Since the present invention have revealed a domain showing cysteine protease inhibitory activity, the above proteins or peptides may be obtained by chemical synthesis based on amino acid sequence including the domain, as well as by gene recombination techniques. For example, appropriate primers are prepared on the basis of the nucleotide sequence coding for the amino acid sequence including the domain. The nucleotide sequence is then amplified by PCR and the like using the primers and cDNA including the target nucleotide sequence as a template. The obtained nucleotide sequence is expressed using an appropriate expression system, thereby the proteins or peptides described above can be obtained.

In general, there exist mutations including substitution, deletion, insertion, addition or inversion of one or plural nucleotides at one or plural positions in a gene, due to difference in species, genus, individual and the like, and substitution arises in an amino acid of a protein encoded by a gene including the above mutation. Lactoferrin and a partial peptide of lactoferrin which can be used for the present invention may thus include such substitution within a range that the cysteine protease inhibitory activity is not impaired.

Lactoferrin or a partial peptide of lactoferrin which can be used for the present invention includes a protein or peptide having amino acid sequence of at least amino acid numbers 679 to 695 of the amino acid sequence shown in SEQ ID No. 1 of the Sequence Listing or a protein or peptide having amino acid sequence of at least amino acid numbers 676 to 692 of the amino acid sequence shown in SEQ ID No. 2 of the Sequence Listing, which includes substitution, deletion, insertion, addition, or inversion of one or plural amino acids and has cysteine protease inhibitory activity. “Substitution, deletion, insertion, addition or inversion of one or plural amino acids” may be set arbitrary as long as it does not influence steric interaction between the protein or the peptide having amino acid sequence of the above-mentioned amino acid numbers and cysteine protease, and does not impair cysteine protease activity. For example, “plural” refers to 2 to 5 amino acids, preferably 2 or 3 amino acid, more preferably 2 amino acids. It varies with a position or a kind of an amino acid residue in the protein conformation of amino acid numbers 679 to 695 of the amino acid sequence shown in SEQ ID No. 1 of the Sequence Listing, or amino acid numbers 676 to 692 of the amino acid sequence shown in SEQ ID No. 2 of the Sequence Listing.

Substitution in the amino acid of amino acid numbers 686 to 690 of the amino acid sequence shown in SEQ ID No. 1 of the Sequence Listing or in the amino acid of amino acid numbers 683 to 687 of the amino acid sequence shown in SEQ ID No. 2 of the Sequence Listing is preferably a substitution which does not change the kind of amino acid. Namely, it is preferable that the replacing amino acids in the above-mentioned sequence are the amino acid having the similar character in the structural classification as the replaced amino acid. Specifically, replacement by aspartic acid, glutamic acid, asparagine or glutamine are preferable when the amino acid in the above-mentioned sequence is acidic amino acid or amide of acidic amino acid, replacement by lysine, histidine or arginine are preferable in the case of basic amino acid, replacement by phenylalanine, tyrosine or tryptophan is preferable in the case of aromatic amino acid, replacement by glycine, alanine, valine, leucine, isoleucine, serine or threonine is preferable in the case of aliphatic amino acid or oxyamino acid, further, in the case of amino acid other than the above-mentioned amino acids (cysteine, methionine, proline etc.), it is preferable that replacement is performed arbitrary as long as it does not impair cysteine protease activity. Further, substitution, deletion and the like of one or plural amino acids may be included in amino acids except the amino acids of amino acid numbers 679 to 695 of the amino acid sequence shown in SEQ ID No. 1 of the Sequence Listing, or amino acids except the amino acids of amino acid numbers 676 to 692 of the amino acid sequence shown in SEQ ID No. 2 of the Sequence Listing. In this case, for example, the term plural refers to 2 to 10 amino acids, preferably 2 to 5 amino acids, more preferably 2 or 3 amino acids, though varying with a position or a kind of an amino acid residue in protein conformation.

Moreover, examples of lactoferrin or a partial peptide of lactoferrin which can be used for the present invention include a protein or a peptide which has homology not less than 80%, preferably not less than 90%, more preferably not less than 95% in an amino acid sequence with a protein or a peptide having amino acid sequence of at least amino acid numbers 679 to 695 of the amino acid sequence shown in SEQ ID No. 1 of the Sequence Listing, or a protein or a peptide having amino acid sequence of at least amino acid numbers 676 to 692 of the amino acid sequence shown in SEQ ID No. 2 of the Sequence Listing, and which has cysteine protease inhibitory activity.

Nucleotide sequence which codes for a protein or a peptide substantially identical with the lactoferrin protein or partial peptide of lactoferrin as described above can be obtained, for example, by modifying the nucleotide sequence with site-directed mutagenesis in such a manner that an amino acid residue located at a specific site includes substitution, deletion, insertion, addition or inversion. The modified nucleotide sequence may be obtained by means of conventional mutagenesis treatment.

Nucleotide sequence which codes for a protein or a peptide substantially identical with lactoferrin or partial peptide of lactoferrin may be obtained by expressing a nucleotide sequence including mutation in appropriate cells and examining cysteine protease inhibitory activity by a method of determining the cysteine protease inhibitory activity described in Test Examples or Examples of the present invention.

In the present invention, transferrin or partial peptide of transferrin may be used. The transferrin used in the present invention may be commercially available transferrin, or transferrin which can be obtained by isolating according to conventional procedures including column chromatography from raw materials including mammalian milk or blood. In addition, transferrin produced by genetic engineering using microorganisms, mammalian cells or transgenic animals etc. can be used.

Metal content in transferrin is not particularly limited for the effect of the cystein protease inhibitor of the present invention, and one or more of the groups consisting of apo-form transferrin which is deironized with hydrochloric acid or citric acid etc., metal-saturated transferrin having saturation degree of 100% which is obtainable by chelating the apo-form transferrin with metals including iron, copper, zinc, manganese etc., and transferrin partially saturated with metal in which metal is bound at saturation degree of less than 100% can be used.

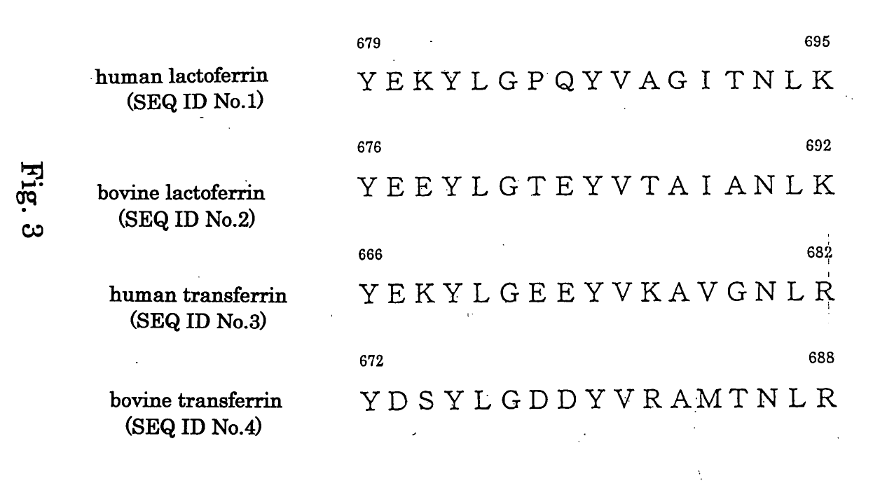

In the present invention, partial peptide of transferrin may be used. The amino acid sequence of transferrin was compared with the amino acid sequences deduced to be the region having cysteine protease inhibitory activity of lactoferrin (the region of amino acid numbers 679-695 of SEQ ID No. 1 or amino acid numbers 676-692 of SEQ ID No. 2), and it was found that the region of amino acid numbers 666-682 (human transferrin peptide Y666-R682) of the amino acid sequence of human transferrin shown in SEQ ID No. 3 and the region of amino acid numbers 672-688 (bovine transferrin peptide Y672-R688) of the amino acid sequence of bovine transferrin shown in SEQ ID No. 4 have high homology with the region having cysteine protease inhibitory activity of lactoferrin (FIG. 3). In addition to this fact, it was demonstrated that transferin have cysteine protease inhibitory activity as shown in the Examples described later, and therefore it was strongly suggested that partial peptides of human transferrin and bovine transferrin having the above-mentioned regions have cysteine protease inhibitory activity.

As production methods of partial peptide of transferrin used in the present invention, there can be exemplified a production method by hydrolyzing the above-mentioned transferrin with acid or protease according to conventional procedures, a production method by producing recombinant peptides according to genetic engineering or a method of producing synthetic peptides according to chemical synthesis. When the production method by hydrolysis is applied, partial peptide of transferrin of the present invention can be used as a mixture in which partial peptide is contained in hydrolysate, or can also be used as a peptide purified according to conventional procedures including HPLC etc.

Preferable forms of transferrin or partial peptide of transferrin used in the present invention can be exemplified as a human transferrin having amino acid sequence shown in SEQ ID No. 3 of the Sequence Listing or a protein or peptide having amino acid sequence of at least amino acid numbers 666-682 of the amino acid sequence shown in SEQ ID No. 3 of the Sequence Listing. There can also be exemplified bovine transferrin having amino acid sequence shown in SEQ ID No. 4 of the Sequence Listing or a protein or peptide having amino acid sequence of at least amino acid numbers 672-688 of the amino acid sequence shown in SEQ ID No. 4 of the Sequence Listing. Note that sequences of amino acid numbers 1 to 19 of the amino acid sequence shown in SEQ ID No. 3 of the Sequence Listing and the amino acid sequence shown in SEQ ID No. 4 of the Sequence Listing are signal sequences.

The peptide consisting of the amino acid sequence of amino acid numbers 666-682 of the amino acid sequence shown in SEQ ID No. 3 of the Sequence Listing and the peptide consisting of the amino acid sequence of amino acid numbers 672-688 of the amino acid sequence shown in SEQ ID No. 4 of the Sequence Listing are considered to have cysteine protease inhibitory activity based on the sequence homology with the amino acid sequence of amino acid numbers 679-695 of SEQ ID No. 1 (human lactoferrin) of the Sequence Listing and the amino acid sequence of the amino acid numbers 676-692 of SEQ ID No. 2 (bovine lactoferrin) of the Sequence Listing, which are revealed to be regions with cysteine protease inhibitory activity in the present invention. Moreover, it is considered that peptides having amino acid sequence which includes amino acid numbers 666 to 682 of SEQ ID No. 3 in the Sequence Listing and further extends to one or both of the N-termius side and the C-termius side, and peptides having amino acid sequences which includes amino acid numbers 672 to 688 of SEQ ID No. 4 in the Sequence Listing and further extends to one or both of the N termius side and the C termius side, have cysteine protease inhibitory activity, because full-length transferrin has cysteine protease inhibitory activity.

Since the present invention have revealed a domain showing cysteine protease inhibitory activity, the above peptides may be obtained by chemical synthesis based on amino acid sequence including the domain, as well as by gene recombination techniques. For example, appropriate primers are prepared on the basis of the nucleotide sequence coding for amino acid sequence including the domain. The nucleotide sequence is then amplified by PCR and the like using the primers and cDNA including the target nucleotide sequence as a template. The obtained nucleotide sequence is expressed using an appropriate expression system, thereby the protein or peptide described above can be obtained.

In general, there exist mutations including substitution, deletion, insertion, addition or inversion of one or plural nucleotides at one or plural positions in a gene, due to difference in species, genus, individual and the like, and substitution arises in an amino acid of a protein encoded by a gene including the above mutation. Transferrin and a partial peptide of transferrin which can be used for the present invention may thus include such substitution within a range that the cysteine protease inhibitory activity is not impaired.

Transferrin or a partial peptide of transferrin which can be used for the present invention includes a peptide having amino acid sequence of at least amino acid numbers 666 to 682 of the amino acid sequence shown in SEQ ID No. 3 of the Sequence Listing or a peptide having amino acid sequence of at least amino acid numbers 672 to 688 of the amino acid sequence shown in SEQ ID No. 4 of the Sequence Listing, which includes substitution, deletion, insertion, addition, or inversion of one or plural amino acids and has cysteine protease inhibitory activity. “Substitution, deletion, insertion, addition, or inversion of one or plural amino acids” may be set arbitrary as long as it does not influence steric interaction between the protein or the peptide having amino acid sequence of the above-mentioned amino acid numbers and cysteine protease, and does not impair cysteine protease activity. For example, “plural” refers to 2 to 5 amino acids, preferably 2 or 3 amino acid, more preferably 2 amino acids. It varies with a position or a kind of an amino acid residue in the protein conformation of amino acid numbers 666 to 682 of the amino acid sequence shown in SEQ ID No. 3 of the Sequence Listing, or amino acid numbers 672 to 688 of the amino acid sequence shown in SEQ ID No. 4 of the Sequence Listing. Substitution in the amino acid of amino acid numbers 673 to 677 of the amino acid sequence shown in SEQ ID No. 3 of the Sequence Listing or in the amino acid of amino acid numbers 679 to 683 of the amino acid sequence shown in SEQ ID No. 4 of the Sequence Listing is preferably a substitution which does not change a kind of amino acid. Namely, it is preferable that the replacing amino acids in the above-mentioned sequence are the amino acid having the similar character in the structural classification as the replaced amino acid. Specifically, replacement by aspartic acid, glutamic acid, asparagine or glutamine are preferable when the amino acid in the above-mentioned sequence is acidic amino acid or amide of acidic amino acid, replacement by lysine, histidine or arginine are preferable in the case of basic amino acid, replacement by phenylalanine, tyrosine or tryptophan is preferable in the case of aromatic amino acid, replacement by glycine, alanine, valine, leucine, isoleucine, serine or threonine is preferable in the case of aliphatic amino acid or oxyamino acid, further, in the case of amino acid other than the above-mentioned amino acids (cysteine, methionine, proline etc.), it is preferable that replacement is performed arbitrary as long as it does not impair cysteine protease activity. Further, substitution, deletion and the like of one or plural amino acids may be included in amino acids except the amino acids of amino acid numbers 666 to 682 of the amino acid sequence shown in SEQ ID No. 3 of the Sequence Listing, or amino acids except the amino acids of amino acid numbers 672 to 688 of the amino acid sequence shown in SEQ ID No. 4 of the Sequence Listing. In this case, for example, the term plural refers to 2 to 10 amino acids, preferably 2 to 5 amino acids, more preferably 2 or 3 amino acids, though varying with a position or a kind of an amino acid residue in protein conformation.

Moreover, examples of transferrin or a partial peptide of transferrin which can be used for the present invention include a protein or a peptide which has homology not less than 80%, preferably not less than 90%, more preferably not less than 95% in an amino acid sequence with a protein or a peptide having amino acid sequence of at least amino acid numbers 666 to 682 of the amino acid sequence shown in SEQ ID No. 3 of the Sequence Listing, or a protein or peptide having amino acid sequence of at least amino acid numbers 672 to 688 of the amino acid sequence shown in SEQ ID No. 4 of the Sequence Listing, and which has cysteine protease inhibitory activity.

Nucleotide sequence which codes for a protein or a peptide substantially identical with the transferrin protein or partial peptide of transferrin as described above can be obtained, for example, by modifying the nucleotide sequence with site-directed mutagenesis in such a manner that an amino acid residue located at a specific site includes substitution, deletion, insertion, addition or inversion. The modified nucleotide sequence may be obtained by means of conventional mutagenesis treatment.

Nucleotide sequence which codes for protein or a peptide substantially identical with transferrin or partial peptide of transferrin may be obtained by expressing a nucleotide sequence including mutation in appropriate cells and examining cysteine protease inhibitory activity by a method of determining the cysteine protease inhibitory activity described in Test Examples or Examples of the present invention.

In the protease inhibitor of the present invention, lactoferrin, a partial peptide of lactoferrin, or transferrin, a partial peptide of transferrin may be used alone, or two or more thereof may be used together. Moreover, one type of a partial peptide of lactoferrin may be used alone, or plural types thereof may be used in combination. Further, one type of a partial peptide of transferrin may be used alone, or plural types thereof may be used in combination.

Lactoferrin, a partial peptide of lactoferrin, or transferrin which can be used for the present invention has inhibitory activity against cysteine proteases such as cathepsin B, L, S and papain. The cysteine protease inhibitory activity can be determined in accordance with the methods of Barrett et al. (Methods in Enzymology, Vol. 80, pp. 535-561, 1981). The method of determination will be described in detail in Test Examples and Examples of the present invention.

The cysteine protease inhibitor of the present invention can be produced by using lactoferrin, a partial peptide of lactoferrin or transferrin, and combining them with a pharmaceutical acceptable carrier. A form of administration unit of the pharmaceutical preparation of the present invention is not particularly limited, and may be appropriately selected in accordance with therapeutic purposes. Specifically, examples of the form of administration unit include a tablet, a pill, powder, liquid, a suspension, an emulsion, granules, capsule, syrup, a suppository, injectable solution, an ointment, a patch, eye-drop and collunarium. Additives such as a vehicle, a bonding agent, a disintegrator, a lubricant, a stabilizer, a flavoring agent, diluent, surfactant and a solution for injection, which are commonly used for pharmaceuticals as a pharmaceutical carrier, may be used upon preparation.

The amount of lactoferrin, a partial peptide of lactoferrin or transferrin contained in the pharmaceutical of the present invention is not particularly limited and may be appropriately selected. For example, it may be usually set to 0.005 to 60% by mass, preferably 0.05 to 50% by mass in the preparation.

Diseases associated with cysteine protease can be treated by oral or parenteral administration of the cysteine protease inhibitor of the present invention to a subject. The term “subject” used herein may be either human beings or mammals other than human.

A method of administering the pharmaceutical of the present invention is not particularly limited, and can be determined according to a form of the pharmaceutical, a subject's age or sex, and other conditions including a degree of the subject's symptom. A dosage of an active ingredient of the pharmaceutical of the present invention can be appropriately set based on a dose regimen, a subject's age or sex, a degree of a disease, and other conditions. Usually, the amount of lactoferrin, partial peptide of lactoferrin or transferrin as active ingredients may be set based on the amount ranging from 0.1 to 1,200 mg/kg/day, preferably 10 to 500 mg/kg/day, and the pharmaceutical may be administered once or plural times per day.

The cysteine protease inhibitor of the present invention is useful as preventive or therapeutic agents for diseases associated with cysteine protease, such as, allergy, muscular dystrophy, myocardial infarct, apoplexy, Alzheimer's disease, multiple sclerosis, cataract, osteoporosis, malignant hypercalcemia, enlarged prostate, breast cancer, prostate cancer, and periodontitis, or as an inhibitor of cancer cell growth or metastasis, or as a growth inhibitor for bacteria (Staphylococcus aureus V8, etc.) or viruses (poliovirus, herpesvirus, coronavirus, AIDS virus, etc.). The cysteine protease inhibitor of the present invention may be used alone, or used in combination with known preventive or therapeutic agents for the above-mentioned diseases or known growth inhibitor of the bacteria or viruses. Such combination can enhance preventive and therapeutic effects against the diseases, or a growth inhibitory effect against the bacteria or viruses. The known preventive and therapeutic agents for the above-mentioned diseases, or known growth inhibitor for the bacteria or viruses to be combined may be contained in the inhibitor of the present invention as an active ingredient, or may be commercialized as another agent and combined upon use without being contained in the inhibitor of the present invention.

The food and drink composition of the present invention may be produced by adding lactoferrin, partial peptide of lactoferrin or transferrin to raw materials for food or drink, and it can be orally ingested. As the raw materials, those employed in drink or food may be generally used. The food and drink composition of the present invention may be prepared in a similar way as usual food and drink composition except that the cysteine protease inhibitor is added. Examples of the forms of the food and drink composition include drinks such as cold drink, carbonated drink, nutrition drink, fruit drink and lactobacillus beverage (including concentrated stock solution and adjustment powder of those drinks); ice sweets such as an ice cream, sherbet and shaved ice; confectionery such as candy, chewing gum, gum, chocolate, confectionery pill, snack food, biscuit, jelly, jam, cream and baked confectionery; milk products such as processed milk, milk beverage, fermented milk and butter; bread; enteral nutrition formula, liquid formula, infant formula and sport drink; and other functional food.

The amount of lactoferrin, partial peptide of lactoferrin or transferrin to be added in the food and drink composition of the present invention is properly set according to the form of the food and drink composition, and normally they may be added in an amount of 0.005 to 60% by mass, preferably 0.05 to 50% by mass in the food or drink.

The feed composition of the present invention can be produced by adding lactoferrin, a partial peptide of lactoferrin or transferrin to feed, and orally administered to general mammals, livestock, pisciculture, farmed fish, and pet animals. Examples of the forms of feed composition include pet foods, livestock feed, and pisciculture feed, and the feed composition of the present invention can be produced by formulating lactoferrin, a partial peptide of lactoferrin or transferrin with grains, lees, rice bran, fish flour, bone manure, oils and fats, skim milk, whey, mineral feed, yeast and the like.

The amount of lactoferrin, a partial peptide of lactoferrin, or transferrin to be added in the feed composition of the present invention is properly set according to the form of the feed composition, and normally they may be added in an amount of 0.005 to 60% by mass, preferably 0.05 to 50% by mass in the feed composition.

The food and drink composition or the feed composition of the present invention may be a food and drink composition or feed composition having indication of its efficacy as preventive or treatment for the diseases shown below. That is, it can be indicated as a preventive or treatment for diseases associated with cysteine protease, such as osteoporosis, malignant hypercalcemia.

The term “indicated” used herein means informing the users of the above-mentioned efficacy, and includes, for example, indicating the above-mentioned efficacy on commercial products of the food and drink composition or the feed composition of the present invention or on packages or advertisement thereof, and transferrin, turning over and displaying the substances having such indication. In particular, an embodiment in which it is indicated as a food for specified health uses [refer to Article 12(1), 5 of the regulation of Health Enforcement Law (Apr. 30, 2003, Ordinance No. 86 of the Japanese Ministry of Health, Labor and Welfare)] is preferable.

Hereinafter, the present invention will be described in detail by showing Test Examples.

### TEST EXAMPLE 1

The present test was carried out to detect cysteine protease inhibitory substance in milk.

(1) Detection Method

The inventor used a technique “reverse zymography” as a method of detecting protease inhibitory substance and detected a protease inhibitory substance located on the gel of SDS-polyacrylamide gel electrophoresis. The reverse zymography is based on a technique opposite of the normal zymography, and the basic principle of the reverse zymography is as follows. That is, a sample containing protease inhibitory substance is applied onto SDS-polyacrylamide gel containing gelatin, and electrophoresis is performed, followed by soaking the gel in a protease solution to degrade protein in the gel. Protease activity is inhibited on a portion where an inhibitory substance is present and gelatin of the portion avoids being degraded by protease and is stained with staining solution, which enables detection of inhibitory substance.

(2) Test Method

A method of the reverse zymography in the present invention is as follows.

Using total milk protein and bovine lactoferrin (manufactured by Morinaga Milk Industry Co., Ltd.) as samples, electrophoresis (hereinafter, SDS-polyacrylamide gel electrophoresis is sometimes abbreviated to SDS-PAGE) was conducted with 12.5% SDS-polyacrylamide gel containing 0.1% of gelatin. After electrophoresis, the gel was washed by immersing it in a 2.5% Triton X-100 solution for 45 minutes, and further washed by repeating three times the operation of immersing the gel in distilled water for 45 minutes. Then, the gel was immersed in 100 ml of a 0.025 M acetic acid buffer solution (pH 5.5) containing 1 mg of papain (31 units/ml), and gelatin was digested by keeping in the solution at 37° C. for 10 hours. The gel was washed with distilled water, stained with a staining solution (0.025% Coomassie brilliant blue (CBB) R-250, 40% methanol, 7% acetic acid aqueous solution) for one hour, and then destained with a destaining solution (40% methanol, 10% acetic acid aqueous solution).

Aside from this, as a control test, the reverse zymography was conducted with a 12.5% SDS-polyacrylamide gel not containing gelatin in the same way as above. Further, typical 12.5% SDS-PAGE (CBB staining) was also performed.

(3) Test Results

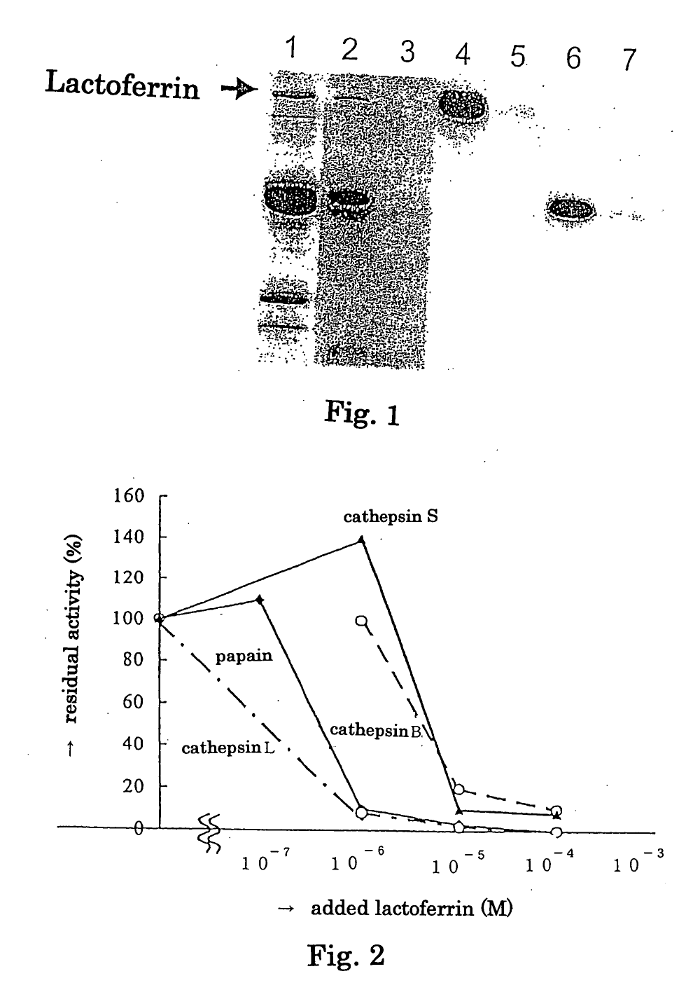

The results of the present test are as shown in FIG. 1. FIG. 1 shows the pattern of the reverse zymography. Lane 1 in FIG. 1 shows a typical SDS-PAGE pattern of the total protein in milk, lane 2 shows a pattern of the reverse zymography of the total protein in milk, lane 3 shows a pattern of the reverse zymography (control), in which the gel does not contain gelatin, of the total protein in milk, lane 4 shows a pattern of the reverse zymography of bovine lactoferrin, and lane 5 shows a pattern of the reverse zymography (control), in which the gel does not contain gelatin, of bovine lactoferrin, respectively. Arrows in the figure show a position of migration of bovine lactoferrin (78 kDa in molecular weight) in SDS-PAGE. On the other hand, lanes 6 and 7 have no direct relations with the present test examples.

As is apparent from FIG. 1, a positive band of the reverse zymography was found in the position almost identical to the position of migration of bovine lactoferrin (78 kDa) in lane 2. This ensured the presence of a substance having cysteine protease inhibitory activity in milk. In addition, a positive band was found in lane 4 which shows the reverse zymography using papain in which bovine lactoferrin was migrated.

The above-described results suggested that lactoferrin derived from bovine has cysteine protease-inhibitory activity.

### TEST EXAMPLE 2

The present test was carried out to determine the N-terminal amino acid sequence of a band of 78 kDa which was suggested to have cysteine protease inhibitory activity in Test Example 1.

(1) Test Method

SDS-PAGE was carried out with total protein sample in milk used in Test Example 1, and proteins were transferred to polyvinylidene difluoride (PVDF) membrane. The PVDF membrane was stained with CBB, and then the stained band migrating in the vicinity of 78 kDa was cut out. The N-termini amino acid sequence of this band was determined with a G1005A Protein Sequencing System, manufactured by Hewlett-Packard Company.

(2) Test Result

As a result of determining the amino acid sequence of the stained band of 78 kDa which was derived from milk, the N-terminal amino acid sequence of the 78 kDa stained band was found to be completely consistent with that of bovine lactoferrin as described in SEQ ID No. 2 of the Sequence Listing. Accordingly, both results of the present test and Test Example 1 revealed that bovine lactoferrin has cysteine protease inhibitory activity.

### TEST EXAMPLE 3

The present test was carried out to determine an inhibitory activity of bovine lactoferrin to cysteine proteases.

(1) Test Method

Inhibitory activities against cysteine proteases including papain, cathepsin B, cathepsin L and cathepsin S (these proteases are available from Wako Pure Chemical Industries, Ltd.) were determined using commercially available bovine lactoferrin (manufactured by Morinaga Milk Industry Co., Ltd.) as a test sample. The inhibitory activity was measured with reference to the method by Barrett et al. (“Methods in Enzymology,” Vol. 80, p 535-561, 1981) as described below. Namely, Z-Phe-Arg-MCA (final concentration of 20 mM: manufactured by Peptide Institute, Inc.) was added as a substrate to 0.1 M acetic acid buffer solution (pH 5.5) in which sample was dissolved at each concentration. Then, cysteine protease (a protease selected from papain, cathepsin B, cathepsin L and cathepsin S in the present test: manufactured by Wako Pure Chemical Industries, Ltd.) solution (final concentration: 15 units/ml) was added, the whole solution was mixed and the reaction was carried out at 37° C. for 10 minutes. Subsequently, fluorescence intensity (excitation wavelength: 370 nm, emission wavelength: 460 nm) of AMC (7-Amino-4-Methyl-Coumarin) released from the digested substrate was measured with a fluorescence spectrometer (manufactured by Hitachi, Ltd.).

(2) Test Result

The results of the present test are as shown in Table 1. Table 1 shows cysteine protease-inhibitory activity of bovine lactoferrin on papain, cathepsin B, cathepsin L and cathepsin S. The results revealed that cysteine protease activities of papain and cathepsin L were completely inhibited by 10⁻⁶ M of bovine lactoferrin, and that cysteine protease activities of cathepsin B and cathepsin S were inhibited more than 70% by 10⁻⁵ M of bovine lactoferrin and completely inhibited by 10⁻⁴ M of bovine lactoferrin. Accordingly, bovine lactoferrin was found to have cysteine protease-inhibitory activity against papain, cathepsin B, cathepsin L and cathepsin S.

TABLE 1

| | cysteine | inhibition rate at each concentration (%) | | | | |
|---|---|---|---|---|---|---|
| inhibitor | protease | 10⁻⁸M | 10⁻⁷M | 10⁻⁶M | 10⁻⁵M | 10⁻⁴M |
| bovine | papain | 0 | 40 | 100 | 100 | 100 |
| lactoferrin | cathepsin L | 0 | 20 | 100 | 100 | 100 |
| | cathepsin B | 0 | 0 | 10 | 70 | 100 |
| | cathepsin S | 0 | 0 | 15 | 80 | 100 |

### TEST EXAMPLE 4

The present test was carried out to determine an inhibitory activity of human lactoferrin to cysteine proteases.

(1) Test Method

Inhibitory activities against cysteine proteases including papain, cathepsin B, cathepsin L and cathepsin S (these proteases are available from Wako Pure Chemical Industries, Ltd.) were determined using commercially available human lactoferrin (manufactured by Morinaga Milk Industry Co., Ltd.) as a test sample, according to the same method as that of determining cysteine protease-inhibitory activity described in the Test Example 3.

(2) Test Results

The results of the present test are as shown in FIG. 2. FIG. 2 shows cysteine protease-inhibitory activity of human lactoferrin to papain, cathepsin B, cathepsin L and cathepsin S. The results revealed that cysteine protease activities of papain and cathepsin L were completely inhibited by 10⁻⁶ M of human lactoferrin, and that cysteine protease activities of cathepsin B and cathepsin S were inhibited more than 80% by 10⁻⁵ M of human lactoferrin and almost completely inhibited by 10⁻⁴ M of human lactoferrin. Accordingly, human lactoferrin was found to have cysteine protease-inhibitory activity against papain, cathepsin B, cathepsin L and cathepsin S.

### TEST EXAMPLE 5

The present test was carried out to determine an inhibitory activity of a partial peptide of lactoferrin to cysteine proteases.

(1) Test Method

Inhibitory activities against cysteine proteases including papain, cathepsin B and cathepsin L (these proteases are available from Wako Pure Chemical Industries, Ltd.) were determined using a peptide having an amino acid sequence of amino acid numbers 679-695 of the amino acid sequence of SEQ ID No. 1 in the Sequence Listing (hereinafter, described as human lactoferrin peptide Y679-K695, FIG. 3) as a test sample, according to the same method as the method of determining cysteine protease-inhibitory activity described in the Test Example 3.

(2) Test Results

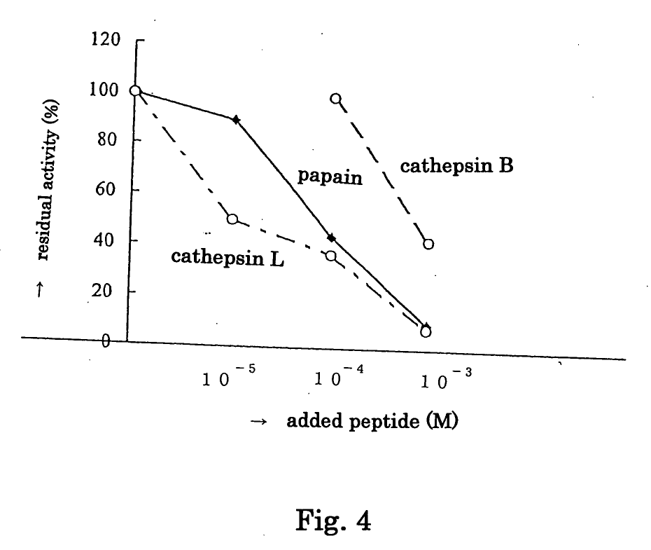

The results of the present test are as shown in FIG. 4. FIG. 4 shows cysteine protease-inhibitory activity of human lactoferrin peptide Y679-K695on papain, cathepsin B and cathepsin L. The results revealed that cysteine protease activities of papain and cathepsin L were inhibited more than 50% by 10⁻⁴ M of human lactoferrin peptide Y679-K695 and almost completely inhibited by 10⁻³ M of human lactoferrin peptide Y679-K695, and that cysteine protease activity of cathepsin B was inhibited nearly 60% by 10⁻³ M of human lactoferrin peptide Y679-K695. Accordingly, human lactoferrin peptide Y679-K695 was found to have cysteine protease-inhibitory activity against papain, cathepsin B and cathepsin L. Further, similar test for determining cysteine protease-inhibitory activity was performed using a peptide having an amino acid sequence of amino acid numbers 676-692 of the amino acid sequence of SEQ ID No. 2 in the Sequence Listing (hereinafter, described as bovine lactoferrin peptide Y676-K692, FIG. 3), and it was found that the bovine lactoferrin peptide Y676-K692 has an activity almost equal to the human lactoferrin peptide Y679-K695.

### TEST EXAMPLE 6

The present test was carried out to determine an inhibitory activity of transferrin to cysteine proteases.

(1) Test Method

Inhibitory activities against cysteine proteases including papain, cathepsin B, and cathepsin L (these proteases are available from Wako Pure Chemical Industries, Ltd.) were determined using commercially available bovine transferrin (manufactured by Nihon Pharmaceutical Co., Ltd.) as a test sample, according to the same method as that of determining cysteine protease-inhibitory activity described in the Test Example 3.

(2) Test Results

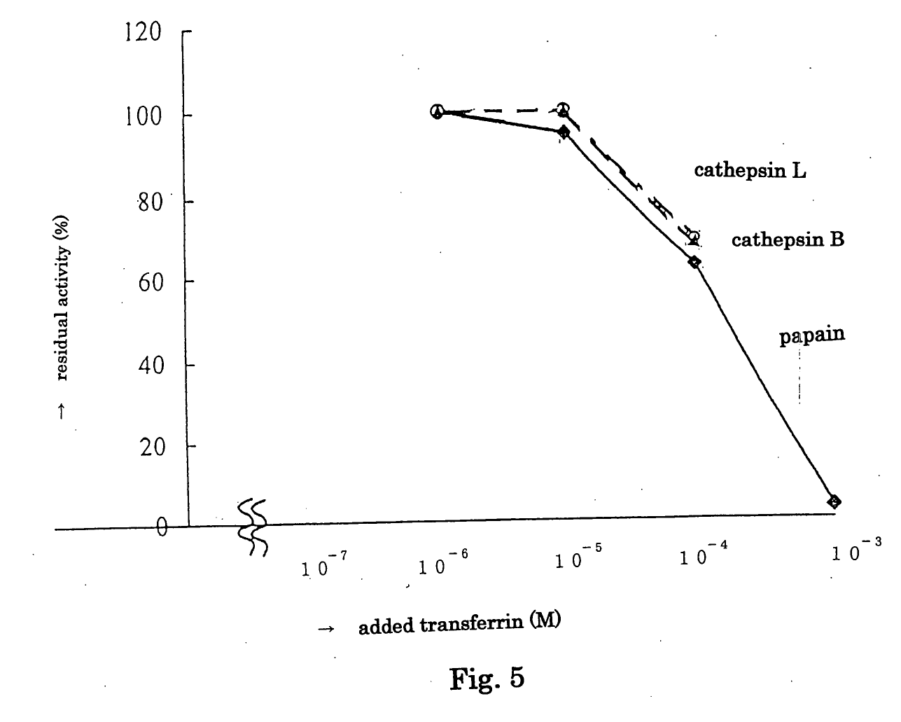

The results of the present test are as shown in FIG. 5. FIG. 5 shows cysteine protease-inhibitory activity of transferrin to papain, cathepsin B and cathepsin L. The results revealed that cysteine protease activity of papain was inhibited 40% by 10⁻⁴ M of transferrin and completely inhibited by 10⁻³ M of transferrin, and that cysteine protease activities of cathepsin B and cathepsin L were inhibited more than 30% by 10⁻⁴ M of transferrin. Accordingly, bovine transferrin was found to have cysteine protease-inhibitory activity against papain, cathepsin B and cathepsin L.

### EXAMPLES

Next, the present invention will be more specifically described by showing examples. However, the present invention is not limited to the following examples.

### Example 1

A peptide having an amino acid sequence of amino acid numbers 679-695 of the amino acid sequence of SEQ ID No. 1 in the Sequence Listing (FIG. 3, human lactoferrin peptide Y679-K695) was produced according to the following procedure.

The above-mentioned peptide of the present invention was produced by synthesizing it with an automatic peptide synthesizer (manufactured by Applied Biosystems Co., Ltd., Model 433A).

Fmoc-group, an amino protective group of HMP resin (manufactured by Applied Biosystems Co., Ltd.) which is a solidified resin for peptide synthesis, was cleaved with N-metylpyrrolidone (manufactured by Applied Biosystems Co., Ltd., hereinafter, abbreviated to NMP) containing 20% of piperidine, and the resin was washed with NMP. Then, Fmoc-threonine [specifically, Fmoc-amino acid (manufactured by Applied Biosystems Co., Ltd.) corresponding to the C-terminal amino acid of the peptide to be synthesized] was condensed to the resin using FastMoc (registered trademark) reagent kit (Applied Biosystems Co., Ltd.), and the resin was washed with NMP. Next, the Fmoc group was cleaved again, and Fmoc-alanine corresponding to the second amino acid from the C-terminus was condensed, followed by washing the resin. Protective peptide resin was prepared by further repeating condensation of Fmoc-amino acid and washing, and crude peptide was recovered from the resin.

Peptides were purified from the crude peptides using high performance liquid chromatography (hereinafter, abbreviated to HPLC). C18-ODS (manufactured by Merck & Co, Inc., Lichrospher 100), a reverse phase column, was used as a column. The obtained purified peptides were analyzed by HPLC to further confirm that the purified product is a single peptide. The amino acid sequence of the purified peptide was determined by use of a gas-phase automatic amino acid sequencer (manufactured by Applied Biosystems Co., Ltd., Model 473A), and it was found to have amino acid sequence of amino acid numbers 679 to 695 in the SEQ ID No. 1.

According to the similar method, a peptide having amino acid sequence of amino acid numbers 676 to 692 in the amino acid sequence described in SEQ ID No. 2 (FIG. 3, bovine lactoferrin peptide Y676-K692) was produced.

### Example 2

### Preparation of a Tablet in Which Lactoferrin is Formulated

A tablet of a cysteine protease inhibitor having the following compositions was produced according to the following procedures.
Bovine lactoferrin (manufactured by Morinaga Milk Industry Co., Ltd.) 40.0(%)
Lactose (manufactured by Morinaga Milk Industry Co., Ltd.) 18.5
Cornstarch (manufactured by Nisshin Flour Milling Co., Ltd.) 30.7
Magnesium stearate (manufactured by Taihei Chemical Industrial Co., Ltd.) 1.4
Carboxymethyl cellulose calcium (manufactured by Gotoku Chemical Co., Ltd.) 9.4

A mixture of bovine lactoferrin, lactose, corn starch and carboxymethyl cellulose calcium was kneaded uniformly while sterile purified water was appropriately added, and the kneaded product was dried at 50° C. for three hours. Magnesium stearate was then added to the obtained dried product and mixed, followed by compression according to a conventional procedure, thereby a tablet was obtained.

### Example 3

### Preparation of Encapsulated Lactoferrin

600 g of lactose (manufactured by Wako Pure Chemical Industries, Ltd.), 400 g of corn starch (manufactured by Nisshin Flour Milling Co., Ltd.), 400 g of crystalline cellulose (manufactured by Wako Pure Chemical Industries, Ltd.) and 600 g of bovine lactoferrin (manufactured by Morinaga Milk Industry Co., Ltd.) were sieved with 50 mesh sieve (manufactured by Yamato Scientific Co., Ltd.), and put into a polyethylene bag with a thickness of 0.5 mm to allow to mix upside down. The obtained powder was packed into a capsule (manufactured by Shionogi Qualicaps, Inc., gelatin capsule No. 1, Op Yellow No. 6 Body, 75 mg of empty weight) in a content of 275 mg using a fully automatic capsule filling machine (manufactured by Cesere Pedini, press type), thereby 7,000 capsules each containing 82 mg of bovine lactoferrin were obtained.

### Example 4

### Preparation of Drink in Which Bovine Lactoferrin is Added

90 g of skim milk (manufactured by Morinaga Milk Industry Co., Ltd.) were dissolved in 800 ml of hot water at a temperature of 50° C., and 30 g of sugar (manufactured by Nissin Sugar Manufacturing Co., Ltd.), 14 g of instant coffee powder (manufactured by Nestle), 2 g of caramel (manufactured by Showa Kako Co., Ltd.) and 0.01 g of coffee flavor (manufactured by San-Ei Gen F.F.I. Inc.) were sequentially added and dissolved in the solution while stirring. Then, the mixture was cooled to 10° C., and 1 g of bovine lactoferrin (manufactured by Sigma Co., Ltd.) was added, thereby milk beverage containing about 0.1% of bovine lactoferrin and having cysteine protease-inhibitory activity was prepared.

### Example 5

### Preparation of Powder of Enteral Nutrition Formula in Which Bovine Lactoferrin is Added

10.8 kg of enzyme degradation product of whey protein (manufactured by Morinaga Milk Industry Co., Ltd.), 36 kg of dextrin (manufactured by Showa Sangyo Co., Ltd.), and a small amount of water-soluble vitamin and minerals were dissolved in 200 kg of water to prepare an aqueous phase in a tank. Aside from this, 3 kg of soybean salad oil (manufactured by Taiyo Yushi Co., Ltd.), 8.5 kg of palm oil (manufactured by Taiyo Yushi Co., Ltd.), 2.5 kg of safflower oil (manufactured by Taiyo Yushi Co., Ltd.), 0.2 kg of lecithin (manufactured by Ajinomoto Co., Inc.), 0.2 kg of fatty acid monoglyceride (manufactured by Kao Corporation) and a small amount of fat-soluble vitamin were mixed and dissolved to prepare an oil phase. The oil phase was added to the aqueous phase in the tank and the whole was mixed by stirring, followed by heating to 70° C. and homogenizing by a homogenizer under a pressure of 14.7 MPa. Next, the resultant was sterilized at 90° C. for 10 minutes, concentrated and spray-dried, thereby approximately 59 kg of powder of intermediate product was obtained. Then, 6.8 kg of sucrose (manufactured by Hokuren-Federation of Agriculture Cooperatives), 167 g of amino acid mixture powder (manufactured by Ajinomoto Co., Inc.) and 60 g of bovine lactoferrin (manufactured by Sigma Co., Ltd.) were added to 50 kg of the powder of the intermediate product, and uniformly mixed, thereby approximately 57 kg of enteral nutrition formula containing bovine lactoferrin and having a cysteine protease-inhibitory activity was obtained.

### INDUSTRIAL APPLICABILITY

As described in detail above, the present invention relates to a cysteine protease inhibitor comprising one or more of lactoferrin, a partial peptide of lactoferrin, and transferrin, as an active ingredient. Effects exerted by the present invention are as follows:
(1) Excellent in safety and capable of being administrated or ingested daily in a long term because it is made of a protein available as food material or a partial peptide thereof.
(2) Having a wide spectrum of inhibitory activity on various cysteine proteases.
(3) Usable as preventive or therapeutic agents for diseases associated with cysteine protease.
(4) Applicable to production of functional food including food for specified health uses and nutritional supplementary food.
(5) Usable as an agent for adjusting physical properties of food in the field of food processing.

1. A cysteine protease inhibitor comprising one or more of lactoferrin, a peptide fragment of lactoferrin, and transferrin, as an active ingredient.

2. The cysteine protease inhibitor according to claim 1, wherein lactoferrin and/or transferrin are one or more of metal-saturated form, partially metal-saturated form and apo form.

3. The cysteine protease inhibitor according to claim 1, wherein lactoferrin and/or a peptide fragment of lactoferrin are one or more of the proteins or peptides selected from the group consisting of:
(a) a protein or peptide having an amino acid sequence of at least amino acid numbers 679 to 695 of the amino acid sequence shown in SEQ ID No. 1 of the Sequence Listing,
(b) a protein or peptide having the amino acid sequence of at least amino acid numbers 679 to 695 of the amino acid sequence shown in SEQ ID No. 1 of the Sequence Listing, with substitution, deletion, addition or inversion of one or plural amino acids, and having cysteine protease inhibitory activity,
(c) a protein or peptide having an amino acid sequence of at least amino acid numbers 676 to 692 of the amino acid sequence shown in SEQ ID No. 2 of the Sequence Listing, and
(d) a protein or peptide having the amino acid sequence of at least amino acid numbers 676 to 692 of the amino acid sequence shown in the SEQ ID No. 2 of the Sequence Listing, with substitution, deletion, addition or inversion of one or plural amino acids, and having cysteine protease inhibitory activity.

4. The cysteine protease inhibitor according to claim 1, which is a preventive or therapeutic agent for a disease associated with cysteine protease.

5. The cysteine protease inhibitor according to claim 4, wherein the disease associated with cysteine protease is osteoporosis or malignant hypercalcemia.

6. A food or drink composition or feed composition, which is produced by adding the cysteine protease inhibitor according to claim 1 to the food or drink composition or feed composition.

7. A method for treating a disease associated with cysteine protease, wherein the cysteine protease inhibitor according to claim 1 is administered to a subject.

8. A method of using one or more of lactoferrin, a peptide fragment of lactoferrin, and transferrin, in manufacture of a cysteine protease inhibitor.

9. The method according to claim 8, wherein lactoferrin and/or transferrin are one or more of metal-saturated form, partially metal-saturated form and apo form.

10. The method according to claim 8, wherein lactoferrin and/or a peptide fragment of lactoferrin are one or more of the proteins or peptides selected from the group consisting of:
(a) a protein or peptide having an amino acid sequence of at least amino acid numbers 679 to 695 of the amino acid sequence shown in SEQ ID No. 1 of the Sequence Listing,
(b) a protein or peptide having the amino acid sequence of at least amino acid numbers 679 to 695 of the amino acid sequence shown in SEQ ID No. 1 of the Sequence Listing, with substitution, deletion, addition or inversion of one or plural amino acids, and having cysteine protease inhibitory activity,
(c) a protein or peptide having an amino acid sequence of at least amino acid numbers 676 to 692 of the amino acid sequence shown in SEQ ID No. 2 of the Sequence Listing, and
(d) a protein or peptide having the amino acid sequence of at least amino acid numbers 676 to 692 of the amino acid sequence shown in SEQ ID No. 2 of the Sequence Listing, with substitution, deletion, addition or inversion of one or plural amino acids, and having cysteine protease inhibitory activity.

11. The method according to claim 8, wherein the cysteine protease inhibitor is a preventive and therapeutic agent for a disease associated with cysteine protease.

12. The method according to claim 11, wherein the disease associated with cysteine protease is osteoporosis or malignant hypercalcemia.


SEQUENCE LISTING (4 sequences)

SEQ ID NO: 1 - protein, 711 aa, Homo Sapiens [sequence omitted]

SEQ ID NO: 2 - protein, 708 aa, Bos taurus
  sig_peptide (1)...(19): Lactoferrin
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 3 - protein, 698 aa, Homo Sapiens
  sig_peptide (1)...(19): Transferrin
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 704 aa, Bos taurus
  sig_peptide (1)...(19): Transferrin
MRPAVRALLACAVLGLCLADPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMAKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP